(19) United States
(12) Patent Application Publication
(10) Pub. No.: US 2002/0086026 A1
(43) Pub. Date: Jul. 4, 2002

(54) Novel vaccine development

(76) Inventor: Andrew William Heath, Sheffield (GB)

Correspondence Address: KLAUBER & JACKSON, 411 HACKENSACK AVENUE, HACKENSACK, NJ, 07601

(21) Appl. No.: 09/938,477

(22) Filed: Aug. 24, 2001

Related U.S. Application Data
(63) Continuation of application No. 08/878,348, filed on Jun. 18, 1997, now abandoned.

(51) Int. Cl. A61K 39/00; C07H 21/04; C12P 21/02; C12N 5/06

(52) U.S. Cl. 424/184.1; 435/69.1; 435/325; 536/23.5

(57) ABSTRACT

The invention relates to a novel antigen that is capable of stimulating the T-cell lymphocyte surface receptor CD40. The adjuvant is a ligand adapted for this purpose. The invention also relates to a novel vaccine incorporating the aforementioned adjuvant and also ideally an antigen that is either a T-cell dependent antigen or a T-cell independent antigen.

The invention relates to a method of manufacture and a system for the production of a novel human or animal vaccine; and also a novel human or animal vaccine.

It is known that the immune system works on the basis of recognition and thus the ability to distinguish between self and non-self. Recognition of non-self, or invading material, is followed by a sequence of steps that are designed to kill or eliminate the non-self material. As knowledge of the immune system grows and molecular biological techniques advance it has become possible to advantageously manipulate the various steps in an immune response in order to enhance the nature of that response. Thus, for example, it has become possible to manufacture a wide range of vaccines using recombinant material and thus manufacture a range of vaccines which were not previously available either because the relevant material was not obtainable or had not before been produced.

The specific immune system is made up of lymphocytes which are able to recognise specific antigens. B lymphocytes recognise antigens in their native conformation through surface immunoglobulin receptors, and T lymphocytes recognise protein antigens that are presented as peptides along with self molecules known as MHC, on the surface of antigen presenting cells. There are a variety of antigen presenting cells including B lymphocytes. T lymphocytes may be further subdivided into cytotoxic T lymphocytes, which are able to kill virally infected “target” cells, and T helper lymphocytes. T “helper” lymphocytes are able to help B lymphocytes to produce specific antibody, or to help macrophages to kill intracellular pathogens.

Bacterial infections caused by encapsulated bacteria are a major world health problem. The species Streptoccocus pneumoniae, Haemophilus influenzae and Neisseria meningitidis are difficult to vaccinate against due to the thymus independent nature of the major surface antigens, the capsular polysaccharides ( ).

T-cell independent antigens present particular problems regarding the development of effective vaccines. Antibody production is low and is not normally boosted by re-immunisation. The antibody isotypes are restricted to the IgM and other isotypes and are generally of a low affinity for a specific antigen.

A major problem lies in the response of young children to T-cell independent vaccines. These individuals are amongst the most vulnerable to the aforementioned bacterial infections. Over 80% of childhood pneumococcal infections occur in infants under the age of two. Coincidentally this age group responds most poorly to T-cell independent antigens.

T-cell dependent antigens are much more effective at eliciting high titre, high affinity antibody responses. This comes about because T lymphocyte help B lymphocytes is elicited during the immune response to these antigens. B lymphocytes binds to antigen through their specific antigen receptors which leads to partial activation. If the antigen is a protein the B lymphocytes take up and process the antigen to peptides which are expressed on the cell surface along with MHC class II molecules. The MHC class II/peptide complex is then recognised by specific T lymphocytes. Upon this recognition the T lymphocytes give “help” to the B lymphocytes, and this “help” along with the initial signal through the antigen receptor results in increased B lymphocyte proliferation, isotype switching and possibly also to increased affinity antibody being eventually produced through somatic hypermutation in the antigen receptor genes. T-cell independent antigens are invariably not protein in composition and cannot therefore be processed and presented by B-lymphocytes via MHC molecules. This failure in antigen presentation results in low T-cell recognition of the antigen thereby resulting in no T-cell help.

T-cell help to B-cells has two components which together with signals through the antigen receptor lead to B-lymphocyte proliferation and antibody production.

1. Cell-cell mediated activation.

2. Cytokine activation.

In vitro experiments have shown that resting B-cells can be stimulated to proliferate after exposure to isolated membranes from activated T-cells. The basis for this phenomenon has been determined. Following T-cell activation a 39kDa (CD154) T-cell specific cell surface protein is induced. This ligand has been identified as the target of the B-cell cell surface receptor CD40 and binding of CD 154 to CD40 is the major component of T lymphocyte help to B lymphocytes.

Further evidence for the involvement of CD40 and CD154 comes from experiments in which host cells transfected with the cDNA encoding the CD154 protein can induce proliferation of B-cells in the presence of added cytokines. In addition, patients with the congenital disease X-linked hyper IgM syndrome, who fail to switch antibody isotypes have been shown to have various mutations in the gene encoding the CD154 protein resulting in failure to activate the B-cells via CD40. The CD40-CD 154 interaction has also been shown to be an important element in immune responses to T-cell dependent antigens in ‘knock-out’ mice.

The other important element in B-cell activation via T-cell help involves cytokine function. Although isolated membranes from activated T-cells can induce B-cell proliferation this effect can be enhanced by the presence of cytolines. Furthermore cytokines have a major role in switching of antibody isotypes. In particular IL4, interferon γ and transforming growth factor beta (TGF β) are of importance. IL4 induces IgG1 and IgE, IFNγ induces IgG2a and TGFB induces IgA and IgG2b. In addition IFNγ is probably responsible for the switching to IgG3 which is seen naturally in responses to T-cell independent antigens. However ligation of CD40 does not induce appreciable Ig secretion on its own, but CD40 ligation (including via T-cell membranes) seems to prepare cells for differentiation which can be induced efficiently by IL4 and IL5.

Finally T-cell help has a major influence on somatic hypermutation which results in the selection of B-cell clones that produce high affinity antibodies.

From this description it may be surmised that T-cell independent production of antibodies by B-cells is compromised due to the lack of help offered by T-helper lymphocytes through activation via CD40 and through the influence of cytokines produced by the T-helper cell.

It is therefore an object of this invention to provide a means of activating B-cells to proliferate and produce the full range of antibody isotypes of high titre in response to T-cell independent, as well as T-cell dependent antigens.

It is a further object of this invention to use T-cell independent and/or dependent antigens to produce effective vaccines that offer high titre, high affinity antibodies to protect individuals from infection.

It is yet a further object of the invention to provide a safe immunological adjuvant for use in a vaccine and also for use in enhancing the immune response to T-cell independent and/or dependent antigens.

It is yet a further object still of the invention to provide a method for the production of a vaccine of the invention.

It is a further object of the invention to provide a system for the production of the vaccine of the invention.

In its broadest aspect the invention concerns the provision of a means for activating the CD40 receptor on a B-lymphocyte, ideally the means comprising an adjuvant which is adapted to activate said receptor, either directly or indirectly. More preferably the invention concerns a ligand which binds to the CD40 receptor on a B-lymphocyte and brings about the activation of same.

According to a first aspect of the invention there is therefore provided an adjuvant which is adapted to stimulate a B-lymphocyte cell surface receptors CD40.

According to a second aspect of the invention there is provided a vaccine suitable for enhancing T-cell independent and T-cell dependent immunity comprising a T-cell dependent and/or independent antigen, or part(s) thereof, and an associated adjuvant which is adapted to stimulate a B-lymphocyte cell surface receptor, CD40.

Reference herein to the term vaccine is intended to include a wide variety of vaccines including, but not limited to, contraceptive vaccines, immunotherapy vaccines and prophylactic or therapeutic vaccines.

Reference herein to T-cell independent immunity includes reference to an immune response which operates wholly or largely independently of T-cells, for example, because existing T-cells are not activated; or because existing T-cells are not functional or immune suppressed through disease or exposure to chemicals, radiation or any other means.

To by-pass or mimic the effects of T-cell help we propose a vaccine which ensures that all B-cells receiving a signal through their specific antigen receptors also receive a signal through CD40, mimicking or improving upon that which would be received during natural T-cell help. This would be achieved, ideally, by ensuring that a CD40 binding moiety were closely associated with the vaccine antigen. This could be through co-administration of the CD40 stimulating moiety with the appropriate T-cell independent and/or dependent antigen, or preferably through covalent linkage, or co-entrapment on/in a carrier system.

The vaccine involves ideally the conjugation of the antigen to a CD40 ligand such as an anti CD40 antibody, or part thereof, followed by immunisation of a human or animal. It should be apparent to those skilled in the art that this methodology may also be applied to any antigens, but in the instance of T-cell dependent antigens could be of particular relevance to those individuals that are immune suppressed and therefore lack T-helper lymphocytes (e.g. AIDS patients).

In a preferred embodiment of the invention said antigen is soluble and ideally a protein or a polysaccharide.

Ideally stimulation of CD40 is via binding of said adjuvant, or part thereof, to at least a part of CD40. In a preferred embodiment of the invention said antigen and adjuvant are bound or cross-linked together.

More preferably said adjuvant is an antibody, either polyclonal or monoclonal, but ideally monoclonal, which is adapted to bind to said CD40. More ideally still said antibody is humanised.

In a preferred aspect of the invention said antibody may be whole or, alternatively, comprise only those domains which are effective at binding CD40 and in particular selected parts of CD40.

In another embodiment of the invention, said adjuvant is a natural ligand of CD40, the T-cell specific CD154 cell surface antigen, ideally produced as a recombinant protein, or a CD40 binding portion of the CD154 protein, or indeed any other ligand, or part thereof, that binds CD40 or part thereof.

In a further embodiment, the CD40 ligand may not be a naturally occurring CD40 ligand but represent an agent that due to its biochemical characteristics has an affinity for CD40.

In its broadest context, reference herein to the term adjuvant includes reference to any string of amino acids or ligand which is selected so as to bind to at least a part of CD40.

In a preferred aspect the recombinant vaccine antigen (when a polypeptide) and the adjuvant will be produced as a chimeric fusion protein.

It will be apparent to those skilled in the art that the said antigen may be a T-cell independent antigen and thus any antigen which is capable of eliciting a T-cell independent response.

Alternatively, the antigen may be a T-cell dependent antigen and thus any antigen that is capable of eliciting a T-cell response.

It is apparent from the above that any antigen may be selected for use in the vaccine of the invention—the precise nature of which will depend on the “disease” that an individual is to be immunised against and/or in some circumstances, the immune status of an individual to be vaccinated.

Ideally said antigen and/or adjuvant is in the form of an immunostimulating complex, or liposomes or biodegradable microspheres, so increasing the association between antigen and CD40 binding moiety.

Alternatively said vaccine comprises an emulsion of the antigen and adjuvant ideally in oil.

In a preferred embodiment of the invention at least one selected cytokine may be included in and/or coadminstered in/with said vaccine.

According to a third aspect of the invention there is provided an adjuvant for enhancing T-cell independent immunity wherein said adjuvant comprises an agent adapted to stimulate a B-lymphocyte surface receptor, CD40.

Preferably said stimulation of said CD40 is via binding of said adjuvant, or part thereof, thereto.

Ideally, said adjuvant is an antibody, either polyclonal or monoclonal, but ideally monoclonal, which is adapted to bind to said CD40. More ideally still said antibody is humanised.

In a preferred aspect of the invention said antibody may be whole or, alternatively, comprise only those domains which are effective at binding CD40, and in particular selected parts of CD40.

In this aspect of the invention said adjuvant is co-administered with either said T-cell independent antigen that is effective at eliciting a T-cell independent immune response or a T-cell dependent antigen that is effective at eliciting a T-cell response. This will be dependent upon the nature of the “disease” against which the individual is to be immnunised and/or the immune status of the individual.

More preferably further still said adjuvant is co-joined to said T-cell independent antigen or said T-cell dependent antigen.

In a yet further preferred embodiment said adjuvant in co-administered with at least one cytokine.

According to a fourth aspect of the invention there is provided a method for the manufacture of a novel vaccine capable of enhancing T-cell independent immunity or T-cell dependant immunity which methods comprises the selection of a suitable T-cell dependant and/or independent antigen, or part(s) thereof, and association or combination of said antigen with an adjuvant wherein said adjuvant is adapted to stimulate a B-lymphocyte receptor, CD40.

According to a fifth aspect of the invention there is provided a method for the manufacture of a novel vaccine capable of enhancing T-cell independent immunity which method comprises the selection of a suitable T-cell dependent and/or independent antigen, or part(s) thereof, and association or combination of said antigen with an adjuvant wherein said adjuvant is adapted to stimulate a B-lymphocyte receptor, CD40.

In yet a further preferred method of the invention said adjuvant is recombinantly manufactured. In yet a further preferred embodiment of the method of the invention said antigen and adjuvant are bound or cross-linked theretogether.

The major T-independent antigens used in vaccines are bacterial capsular polysaccharides. In a preferred embodiment or method of the invention one will therefore purify polysaccharide antigens and crosslink them to a CD40 binding moiety. A commonly used technique for the cross linking of polysaccharide to protein is carbodiinide coupling. However a number of heterobifunctional cross-linking agents are commercially available for both protein-protein and protein-carbohydrate cross-linking. Heterobifunctional cross-linking agents have the advantage that they favour protein-carbohydrate cross-links thereby maximising the yield of adjuvant coupled to antigen.

Preferably said stimulation of said CD40 is via binding of said adjuvant, or part thereof, thereto.

Ideally, said adjuvant is an antibody, either polyclonal or monoclonal, but ideally monoclonal, which is adapted to bind to said CD40. More ideally said antibody is humanised.

In a preferred aspect of the invention said antibody may be whole or, alternatively, comprised only those domains which are effective at binding CD40, and in particular selected parts of CD40.

In a preferred method of the invention one adds at least one cytokine to said vaccine.

According to a further aspect of the invention there is provided a system for the manufacture of a vaccine capable of enhancing T-cell independent or T-cell dependent immunity which system comprises a cell expressing a selected T-cell dependent and/or independent antigen, or part(s) thereof, and also an adjuvant capable of stimulating a B-lymphocyte receptor, CD40.

According to a yet further aspect of the invention there is provided a system for the manufacture for a vaccine capable of enhancing T-cell independent immunity which system comprises a cell expressing a selected T-cell dependent or independent antigen, or part(s) thereof, and also an adjuvant capable of stimulating a B-lymphocyte receptor, CD40.

More preferably still both said antigen (when a polypeptide) and said adjuvant are adapted so as to be secreted from said cell. This may be undertaken by providing both the antigen and adjuvant with secretion signals or providing for the production of a single piece of material comprising both the antigen and the adjuvant and having a single secretion signal associated therewith. It will be evident that in the former instance the said antigen and adjuvant will be found in associated or unbound or uncross-linked manner in the supernatant of the system and in the latter instance said antigen and adjuvant will be co-joined in the supernatant of the system.

Preferably said stimulation of said CD40 is via binding of said adjuvant, or part thereof, thereto.

Ideally, said adjuvant is an antibody, either polyclonal or monoclonal but ideally monoclonal, which is adapted to bind to said CD40. More ideally said antibody is humanised.

In a preferred aspect of the invention said antibody may be whole or, alternatively comprise only those domains which are effective at binding CD40, and in particular selected parts of CD40.

It will be apparent from the above that the invention is based upon the realisation that immune responses, whether to a T-cell independent or a T-cell dependent antigen, can be enhanced by stimulating the B-cell CD40 receptor using any suitable means.

According to a yet further aspect of the invention there is provided a nucleic acid molecule encoding any one or more of the aforementioned embodiments of the invention.

In this last aspect of the invention said nucleic acid molecule may be administered, conventionally, to an individual or animal to be treated so that the adjuvant and ideally also the antigen of the vaccine may be manufactured in vivo.

An embodiment of the invention will now be described by way of example only with reference to the following figures wherein:

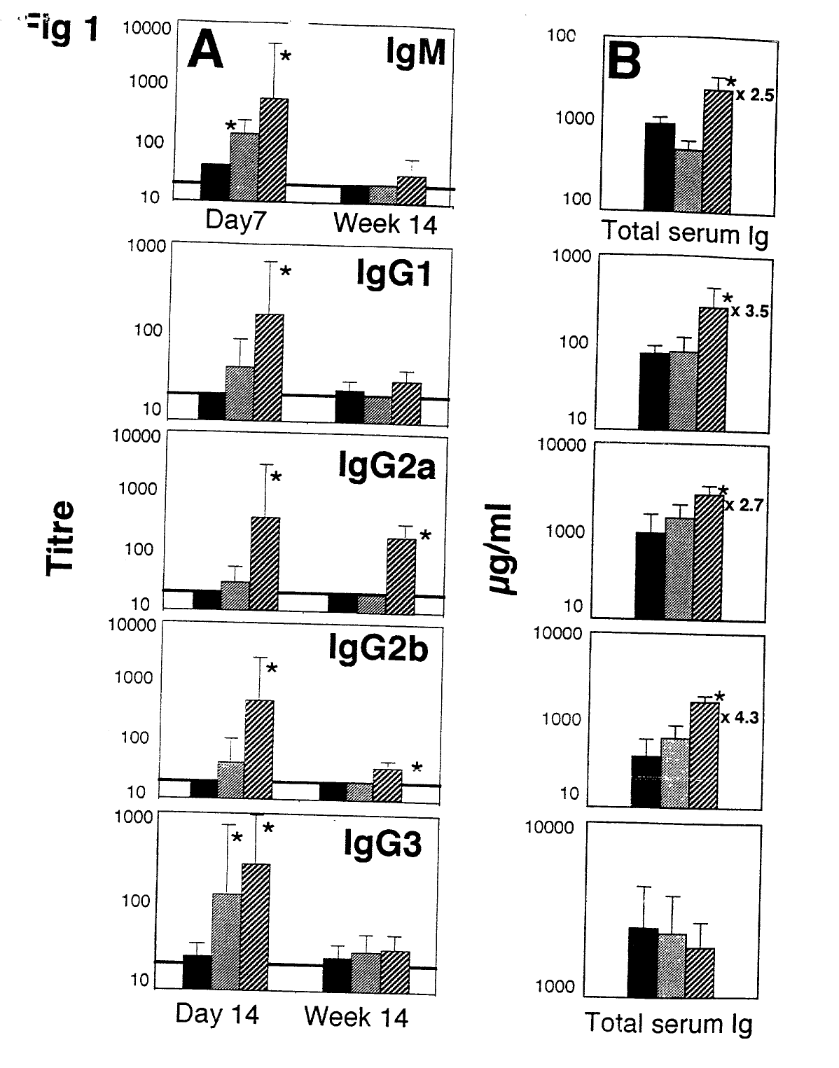

FIG. 1: Shows CD40 antibody induced enhanced, class switched antibody responses to PS3 (type 3 pneumococcal polysaccharide) (A) and increased total serum immunoglobulin (B). BALB/c mice (6-10 weeks old) were injected i.p with 20 ng of PS3 and 500 μkg of 1C10, 4F11 (anti-mouse CD40) or isotype control antibody GL117. Sera were obtained days 7, 14 and week 14 after injection. The IgM and IgG isotype mean logarithmic titres are shown when they were maximal, respectively, day 7 and day 14 after injection. All negative results were given a logarithmic titre of 20, the lowest dilution used. * indicates statistical significance compared with the relevant GL117 control (Student's T test p<0.05).

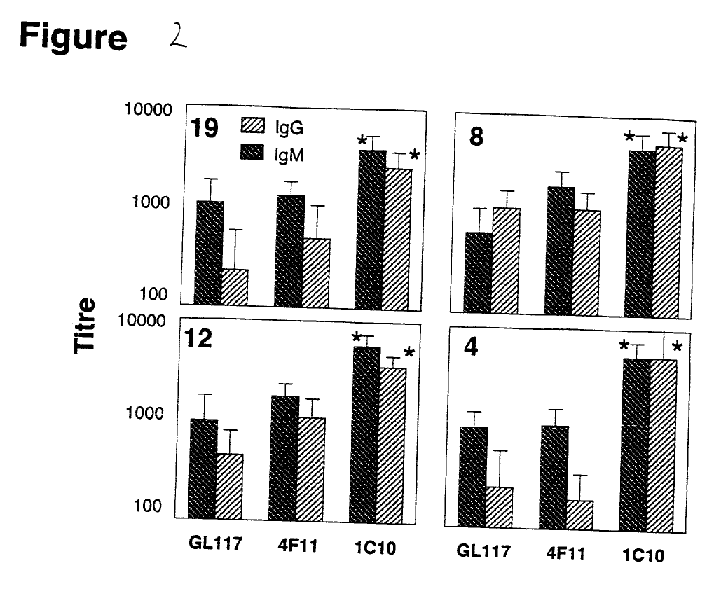

FIG. 2: Shows antibody responses to other pneumococcal polysaccharides are also enhanced by CD40 antibody. IgM and IgG responses to types 8, 4, 12 and 19 S. pneumoniae capsular polysaccharides in mice immunised with the 23 capsular polysaccharides in Pneumovax II (Merck Sharp and Dohme, USA) and either the CD40 antibodies 4F11, 1C10 (anti-mouse CD40) or control antibody GL117. Groups of five BALB/c mice, were injected i.p with either 500 μg of 1C10, 4F11 or GL117, and 1/25th of the recommended human dose of Pneumovax II (commercial 23-valent pneumococcal polysaccharide vaccine, 1 μg each of the 23 polysaccharides present). Sera were obtained on day 10 after injection. All negative results were given a logarithmic titre of 20, the lowest dilution used. All the 1C10 responses were significantly different from the GL117 responses (Student's T test p<0.05).

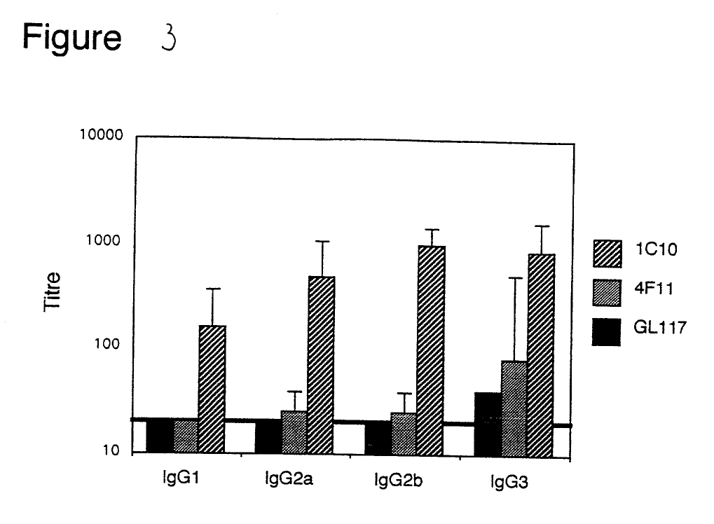

FIG. 3: Shows that the mechanism of 1C10 action is CD4+ cell independent. PS3 specific antibody logarithmic titres induced in CD4 depleted BALB/c mice treated i.p with 20 ng of PS3 and 500 μg of 1C10, 4F11 or control antibody GL117. These mice failed to respond to co-administered keyhole limpet haemocyanin nor were any CD4+ splenocytes discemable on FACS by FITC anti CD4 (data not shown). Sera were obtained on day 14 after injection. All negative results were given a logarithmic titre of 20, the lowest dilution used. All 1C10 responses were significantly different from the relevant GL117 control (Student's t test p<0.05).

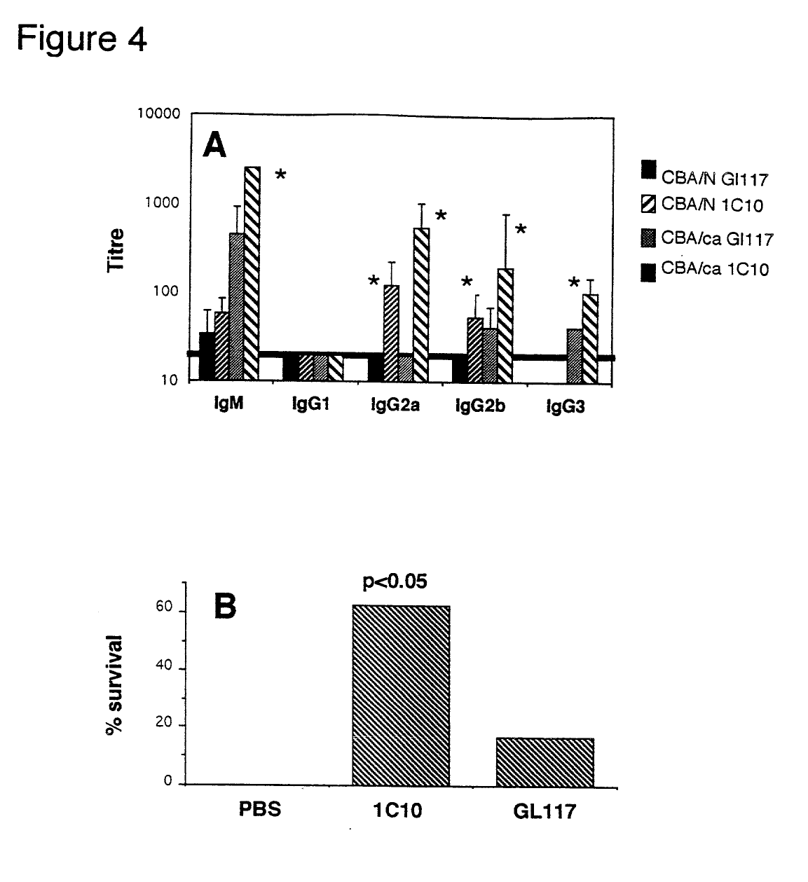

FIG. 4: Shows CD40 antibodies induce responses to PS3 in normally unresponsive xid mice (A). Enhanced responses in BALB/c mice provide protection against S. pneumoniae challenge 9 months after treatment (B). (A) PS3 specific antibody responses in CBA/N(xid) mice injected with 20 ng of PS3 and 1C10, GL117 and/or control CBA/ca mice with 1C10 and GL117. The IgM and IgG isotype logarithmic titres shown are when they were maximal, respectively, day 7 and day 14 after injection. All negative results were given a logarithmic titre of 20, the lowest serum dilution used. * indicates statistical significance compared with the relevant GL117 control (Student's T test p<0.05). B) Percentage survival in BALB/c mice challenged with S. pheumoniae type 3, but administered 9 months previously with 20 ng PS3 and 500 μg of 1C10, GL117 or PBS. Survival in the 1C10 group was significantly enhanced compared to the control groups (p<0.05χ² test).

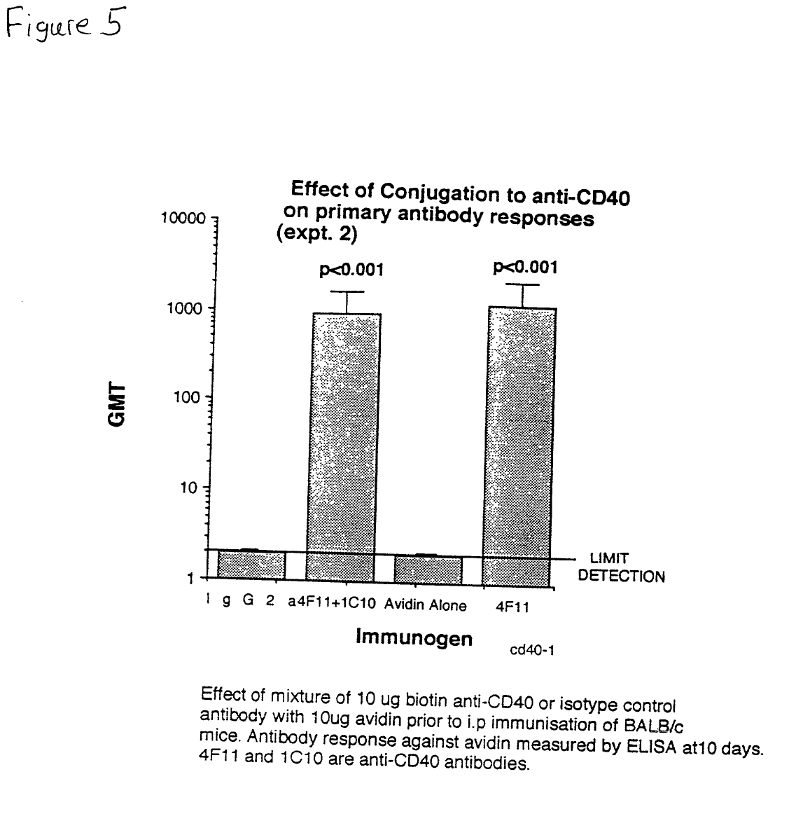

FIG. 5: Shows primary antibody responses to avidin conjugated to biotinylated CD40 antibodies are enhanced. BALB/c mice were immunized with either 10 μg of control IgG2a, 10 μg of avidin conjugated to anti CD40 monoclonal antibody 4F11, 1C10 μg of a combination of avidin conjugated to anti CD40 antibodies 4F11 and 1C10 or 10 μg of non-conjugated avidin. Antibody responses against avidin were measured by ELISA at 10 days post-immunisation.

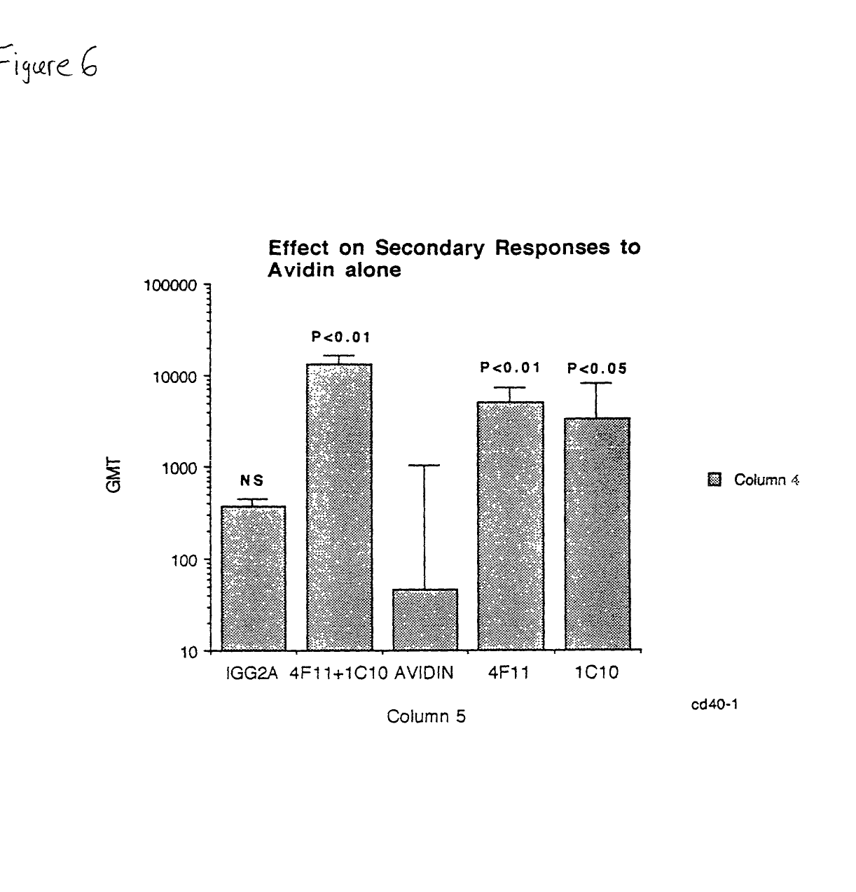

FIG. 6: Shows secondary antibody response to avidin alone following primary immunisation with avidin conjugated to anti CD40 antibodies 4F11 and 1C10. Experimental details are essentially as described in FIG. 5, except that mice received an immunisation with 10 μg avidin alone one month after primary immunisation as in FIG. 5, mice were bled 10 days after this second injection and antibody responses measured by ELISA.

Methods

Mice and Materials

The mice used were BALB/c mice (in house), CBA/ca and CBA/N (xid) mice (Harlan-Olac). They were 6-12 weeks old at the start of the experiments. The pneumococcal capsular polysaccharides type 1, 3, 4, 8, 12, 13, 19 and 23 were obtained from ATCC, USA, pneumococcal cell wall polysaccharide from Statens Serum Institute, Denmark and Pneumovax II vaccine from Merck Sharp and Dohme, USA. Avidin was purchased from Sigma (Poole, Dorset). Biotinylated and non-biotinylated anti-CD40 antibodies were purified from hybridoma supernatants in house and biotinylated in house where necessary using standard reagents (Pierce).

Immunisation Protocols

Mice were treated with 500 μg of either 1C10, 4F11 or GL117 and 20 ng of PS3 i.p. except those receiving Pneumovax II. BALB/c mice receiving Pneumovax II were injected i.p. with either 500 μg of 1C10 or GL117 and 1/25th of the recommended human dose of Pneumovax II. This equates to 1 μg of each of the 23 polysaccharides present in the vaccine. At least 5 mice were used for each experimental group. In experiments where mice were immunised with avidin conjugated to biotinylated anti-CD40, avidin at 1 mg/ml and biotinylated antibody at 1 mg/ml were mixed together at a 1:1 ratio and left on ice for 30 minutes. The conjugates were then diluted in PBS to give a total of 10 μg antibody and 10 μg avidin in 0.2 ml PBS, which was then injected intraperitoneally. In cases where avidin alone was used it was pre-mixed with an equal volume of PBS and left on ice for 30 minutes before dilution and injection.

Experiment in CD4 Depleted Mice

BALB/c mice, 6-10 weeks old, were depleted of CD4 cells 5 days before the experiment start. 500 μg of depleting anti CD4 antibody YTS 191.1 was injected intravenously and again the next day intraperitoneally. The percentage of CD4+ splenocytes in the depleted mice as detected by flow cytometry had dropped to undetectable levels when the antibody and PS3 were injected. There was no antibody response to 50 μg to keyhole limpet haemocyanin, a T dependent antigen, co-administered with the PS3 (data not shown).

Measurements of Polysaccharide Antibodies and Total Serum Immunoglobulin by ELISA

96 well ELISA plates (Costar, UK) were coated overnight with 10 μg/ml polysaccharide or with a 1/200 dilution of anti mouse Ig serum (Sigma, UK). Individual sera were titrated on the plates and the various isotypes detected by HRP conjugated mouse isotype specific sera (Southern Biotechnology Associates, USA). Sera obtained from mice injected with Pneumovax II were absorbed against S. pneumoniae cell wall polysaccharide as described previously. Antibodies to cell wall polysaccharide, a contaminant of all capsular polysaccharide preparations might have created false positive results. Total serum immunoglobulin concentrations were calculated with reference to calibrated mouse serum (Sigma, UK). With the polysaccharide results end point titres for each mouse were assessed against normal mouse serum and then geometric mean titres and standard deviation calculated.

Measurement of Anti-Avidin Responses by ELISA

96 well ELISA plates (Costar, UK) were coated overnight with 10 μg/ml avidin (Sigma) in PBS. After blocking for 1 hour with 3% bovine serum albumin individual sera were titrated on the plates, incubated at room temperature for 1 hour, and following washing, antibody was detected using HRP conjugated anti-mouse immunoglobulin (Southern Biotechnology Associates USA), and substrate (OPD Sigma). End point titres for each mouse were assessed against normal mouse serum, and then geometric mean titres and standard deviation calculated.

Challenge with S. Pneumoniae

BALB/c mice were immunised 9 months before challenge with 20 ng PS3 and 500 μg 1C10 i.p. Challenge was 10⁵ colony forming units of encapsulated S. pneumaniae type 3 (ATCC) given i.p. Final numbers surviving were ascertained 2 weeks after challenge.

Results and Discussion

The development of vaccines against encapsulated bacteria, such as Streptococcus pneumoniae, Haemophilus influenzae and Neisseria meningitidis, is centred on their distinctive capsular polysaccharides. Unfortunately, the inability of antigen presenting cells (APC) to process and present polysaccharides with MHC class II means that these antigens cannot stimulate T-cells. Polysaccharide specific B-cells receive no direct help from their T-cells and, therefore, these antigens are considered T independent (TI-II). Due to this lack of help, TI-II antibody responses are of low titre, low average affinity, and are predominantly of the IgM class with no boosting on second or later exposures to antigen. The T-cell help provided during immune responses to TD antigens induces high titre and isotype switched antibody responses. The major stimulus to B-cells is provided by CD154 (formerly CD40 ligand or gp39), which is expressed de novo on activated T-cells. The CD154 molecule binds the CD40 antigen, which is constitutively expressed on B-cells, and their interactions provide key signals as immune responses develop. CD40 activation is important for the initiation of B-cell proliferation, immunoglobulin class switching, germinal centre responses, and the production of memory B-cells and plasma cells. B-cells responding to TI-II antigens lack T-cell derived cytokines and CD40 ligation and produce, as a result, the poor antibody response characteristic of TI-II antigens. We have investigated in vivo whether the administration of pneumococcal polysaccharide with anti-mouse CD40 antibody could provide a substitute for CD154 mediated CD40 ligation. The two antibodies used were 1C10 and 4F11, chosen they are both rat IgG2a anti-mouse CD40 antibodies but possess markedly different in vitro properties.

Intraperitoneal immunisation of BALB/c mice with type 3 pneumococcal capsular polysaccharide (PS3) alone induced weak IgM and IgG3 responses against the antigen (FIG. 1A). This is typical of the response to TI type II antigens in mice (humans produce IgM and IgG2). Administration of antibodies 1C10 or 4F11 with PS3 induced small but significant rises in specific IgM and IgG3, while remarkably, 1C10 induced significant polysaccharide specific IgG1, IgG2a and IgG2b responses. These isotypes are not normally seen in response to TI II antigens. 1C10 would appear to have successfully mimicked T-cell help by inducing high antibody titres and isotype switching in vivo. The anti-polysaccharide response was extremely persistent, with antibody being detected at high titres 14 weeks after the single immunisation (FIG. 1A). No memory response against the polysaccharide was induced as a second injection of polysaccharide alone failed to boost antibody responses (data not shown).

S. pneumoniae has over 80 different capsular polysaccharide types and any vaccination would be expected to induce protective immunity against a number of the more common serotypes. A current pneumococcal vaccine, Pneumovax II (Merck, Sharp and Dohme), consists of 23 different polysaccharides. Mice were immunised with this 23-valent vaccine and 1C10. FIG. 2 shows that inclusion of the CD40 antibody successfully generated strong IgG responses against randomly chosen polysaccharide types 4, 8, 12 and 19. Such isotype switched responses were also generated against the two other antigens we examined, types 3 and 14 (data not shown). Therefore, 1C10 enhances responses to TI-II antigens other than just PS3.

Given that administration of CD40 antibody mixed with polysaccharide would not restrict or even target CD40 ligation to antigen specific B-cells, we anticipated polyclonal activation of B-cells with a resultant rise in total serum immunoglobulin levels. Indeed 1C10 and PS3 induced some splenomegaly and 2-4 fold rises in total serum immunoglobulin levels (FIG. 1B). This, however, should be contrasted with up to 5-fold rises in specific antibody levels, indicating that polysaccharide specific antibody production was preferentially enhanced. This skewing towards specific antibody is also not unexpected as it reflects in vitro findings. In vitro, while 1C10 could induce B-cell proliferation in the absence of stimulation through the antigen receptor, proliferation was synergistically enhanced by such co-stimulation. 4F11, which largely lacks agonist activity in vitro, did not enhance responses as efficiently as 1C10, demonstrating an association between adjuvant activity in vivo and B-cell activation in vitro.

CD40 ligation is necessary for switching to IgG isotypes during a T dependent response, but various cytokines also play important roles. It was, therefore, intriguing that such isotype switched responses were obtained without the addition of exogenous cytokines. This suggests either that CD40 and antigen receptor ligation may be sufficient to induce isotype switching or that bystander cells may provide sufficient cytokines to switch the activated B-cells in vivo. We considered that the CD40 antibodies might be stimulating T-cell production, either directly through ligation of CD40 on T-cells or indirectly through induction of co-stimulatory molecules on B-cells or other APCs. The action of 4F11 showed T-cell dependency as it failed to augment polysaccharide specific responses in CD4 depleted mice (FIG. 3). However, 1C10 and PS3 administration induced a pronounced, isotype switched response in CD4 depleted mice (FIG. 3) with IgG responses to polysaccharide being better than those induced in normal mice, demonstrating a CD4 independent action. Similar results were obtained when athymic nude mice were used instead of CD4 depleted mice (data not shown).

Most vaccines under development for use against encapsulated bacteria are protein-polysaccharide conjugates which aim to provide T-cell help for the anti-polysaccharide response through T-cell recognition of epitopes on the protein. By their nature such conjugates are not as effective in CD4 deficient patients such as those with AIDS. In contrast the use of a CD40 stimulator would not only avoid the high cost of conjugate production, but as we have shown, generate responses unaffected by a CD4 deficiency.

The major fault with capsular polysaccharide only vaccines is that infants and young children, whilst reacting normally to TD antigens, respond poorly to TI-II antigens. Indeed children under two years old fail to respond at all to many TI-II antigens. The inability of their immune systems to act against bacterial capsules correlates with increased susceptibility to infection. They are the group most in need of effective vaccines. CBA/N (xid) mice have an X-linked immunodeficiency rendering them, like infants, unable to respond to TI-II antigens. Although one report has stated otherwise, in our hands these mice react normally to CD40 ligation in vitro (and unpublished data A. H.). We immunised groups of xid mice with 1C10 plus PS3 and successfully generated IgG2a and IgG2b responses against PS3 (FIG. 4A). Thus, the B-cell defect in these mice was successfully by-passed by administering the CD40 antibody as an adjuvant along with antigen.

Using the mouse model system, we have shown that CD40 simulators can enhance the antibody response to pneumococcal polysaccharides, producing greater antibody levels and the production of IgG isotypes. Similar to protein-polysaccharide conjugates, 1C10 can induce polysaccharide specific responses in xid mice, which like infants are unable to respond to polysaccharide only based vaccines. Unlike protein-polysaccharide conjugates, the adjuvant action of 1C10 is CD4 cell independent, which is a definite advantage for the vaccination of patients with CD4 deficiencies, for example AIDS sufferers.

While 1C10 administered with PS3 clearly enhances specific antibody responses, the measure of a vaccine is whether it provides long-term protection against disease. We challenged mice, immunised 9 months previously, with 10⁵ CFU of S. pneumoniae type II (FIG. 4B). Of the BALB/c mice administered with PS3 and 1C10 five of eight survived challenge, whereas only one of six and none of eleven mice survived in the groups receiving, respectively PS3 with GL117 and PS3 alone (p<0.05χ²test).

Finally, the induction of polyclonal antibody responses, as previously described in FIG. 1B, may increase the risk of auto antibody production. We have investigated this problem by reducing the need to administer elevated doses of anti CD40 antibody by conjugating biotinylated anti CD40 antibody with avidin (a natural ligand of biotin). By physically linking the adjuvant and antigen we have been able to reduce adjuvant levels by approximately 50-fold. FIG. 5 shows the primary antibody responses of BALB/c mice to a combination of biotinylated 4F11 and 1C10 conjugated with avidin, to biotinylated 4F11 conjugated to avidin or to avidin alone. The primary antibody response to avidin is comparable to the response to avidin plus biotinylated IgG2a control antibody. However significant enhancement of antibody levels to avidin is achieved in response to immunisation with a biotinylated anti CD40/avidin conjugate. FIG. 6 shows secondary antibody responses. Clearly the physical linkage of antigen to adjuvant leads to enhanced antibody responses to avidin with a reduction in the amount of adjuvant required. This methodology may also be applied to T-cell independent antigens like the capsular polysaccharides of S. pneumoniae. Techniques for conjugating polysaccharides to protein do exist and will allow this strategy to be further developed.

It is evident that CD40 simulators, such as antibodies, recombinant soluble CD154, or molecular mimics of CD154, have considerable potential as immunological adjuvants for T-cell dependent/independent antigens.

1. An adjuvant which is adapted to stimulate a B-lymphocyte cell surface receptor, CD40.

2. A vaccine including the adjuvant according to claim 1.

3. A vaccine according to claim 2 wherein said vaccine comprises a T-cell dependent and/or T-cell independent antigen, or part(s) thereof.

4. A vaccine according to claim 2 wherein said adjuvant is a CD40 ligand, or part thereof.

5. A vaccine according to claim 2 wherein said adjuvant is an antibody raised against said CD40 receptor, or a part thereof.

6. A vaccine according to claim 5 wherein said antibody is monoclonal.

7. A vaccine according to claim 5 wherein said antibody is humanised.

8. A vaccine according to claim 3 wherein said antigen is soluble.

9. A vaccine according to claim 3 wherein said antigen is a protein.

10. A vaccine according to claim 3 wherein said antigen is a polysaccharide.

11. A vaccine according to claim 3 wherein said adjuvant and antigen are joined theretogether.

12. A vaccine according to claim 11 wherein said antigen is a protein or part thereof, and it is fused to said adjuvant so as to provide a fusion protein.

13. A vaccine according to claim 2 comprising at least one cytoline.

14. A vaccine according to claim 2 suitably formulated from administration to an individual or animal to be vaccinated.

15. A method for the manufacture of a novel vaccine capable of enhancing immunity which method comprises the selection of a suitable T-cell dependent and/or T-cell independent antigen, or part(s) thereof, and association or combination of said antigen with an adjuvant wherein said adjuvant is adapted to stimulate B-lymphocyte receptor, CD40.

16. A method according to claim 15 wherein said vaccine is capable of enhancing T-cell independent immunity.

17. A system for the manufacture of a vaccine capable of enhancing T-cell independent or T-cell dependent immunity which system comprises a cell expressing a selected T-cell dependent and/or T-cell independent antigen, or part(s) thereof, and also an adjuvant capable or stimulating a B-lymphocyte receptor, CD40.

18. A system according to claim 17 wherein said vaccine is capable of enhancing T-cell independent immunity.

19. A system according to claim 17 wherein one or both of said antigen and adjuvant is provided with a secretion signal whereby expression of one or both of said antigen or adjuvant results in secretion of one or both of said antigen or adjuvant from said cell.

20. A system according to claim 17 wherein the expression of said antigen and adjuvant is adapted such that a single fusion protein is manufactured by said cell.

21. A system according to claim 20 wherein said single fusion protein is adapted for secretion from said cell.

22. A nucleic acid molecule encoding an adjuvant according to claim 1 or a vaccine according to claim 2.
